(19) United States
(12) Patent Application Publication
(10) Pub. No.: US 2026/0335184 A1
(43) Pub. Date: Oct. 29, 2026

(54) ELECTROSTATIC ACUPUNCTURE ANESTHESIA DEVICE

(71) Applicant: Jianan Wang, Shenzhen, Guangdong (CN)

(72) Inventor: Jianan Wang, Shenzhen, Guangdong (CN)

(21) Appl. No.: 19/117,632

(22) PCT Filed: Oct. 12, 2023
(86) PCT No.: PCT/CN2023/124141

§ 371 Date: Apr. 1, 2025

(30) Foreign Application Priority Data
Nov. 11, 2022 (CN) 202211408806.5

(51) Int. Cl. A61H 39/00; A61H 39/08

(52) U.S. Cl. CPC A61H 39/002; A61H 39/086; A61H 2201/169; A61H 2203/03

(57) ABSTRACT

Disclosed is an electrostatic acupuncture anesthesia device, comprising: at least one conductive needle which can be pierced into the acupoint or blood vessel of a human or animal body, wherein further includes an electrostatic generator; the conductive needle is in conductive connection with the electrostatic generator, so that the electrons generated by the electrostatic generator can be released in the punctured acupoint of the human or animal body through the conductive needle. The electron generated by the electrostatic generator is used to reduce the concentration of sodium and potassium ions outside the nerve cell membrane in the acupuncture acupoints of the patient, so as to block the transmission of nerve impulse and achieve the purpose of pain relief.

### TECHNICAL FIELD

The invention relates to an acupuncture device, and more particularly to an electrostatic acupuncture anesthesia device for enhancing the analgesic effect during acupuncture anesthesia.

### BACKGROUND ART

The current hand needle acupuncture anesthesia is performed by the anesthesiologist with bare hands (without gloves) through continuous twisting and repeatedly lifting and inserting metal acupuncture that has been pierced into the patient's special acupoints. Because the bare hand (without gloves) twisting the metal needle ensures that the metal needle is electrically connected to the anesthesiologist's hand, and because the human body is equivalent to a capacitor, the physics of acupuncture anesthesia is achieved by inserting conductive acupuncture needles into the patient's specific acupoints to neutralize the charge of charged ions that collect in the patient's acupoints. If the patient's acupoint is negatively charged, the conductive acupuncture needle will export electrons from the patient's acupoint to the anesthesiologist's body to neutralize the negative charge of the patient's acupoint; If the patient's acupoint is positively charged, the conductive acupuncture needle will export electrons from the anesthesiologist's body to the patient's acupoint to neutralize the positive charge of the patient's acupoint.

Because when a nerve cell is at rest (the state when the cell is not stimulated is called the resting state, and the electrical property at this time is the resting potential), the outside of the nerve cell membrane is positively charged, and the inside of the membrane is negatively charged. After the acupuncture needle is inserted into the acupoint, the positively charged ions such as sodium ions and potassium ions outside the nerve cell membrane in the acupoint will be slowly neutralized by the electrons transported from the patient's body and the acupuncturest's body through the acupuncture needle, resulting in a decrease in the concentration of sodium ions and potassium ions outside the nerve cell membrane in the acupoint (but the concentrations of sodium and potassium elements remain unchanged).

The reduction of sodium ion concentration can lead to a decrease in the action potential (action potential is a rapid, reversible, and propagable electrical change on both sides of the cell membrane that occurs in excitable tissues or cells when they receive a suprathreshold stimulus, based on the resting potential. The main component of the action potential is the peak potential. The action potential can be divided into three processes: depolarization, repolarization, and hyperpolarization. The generation of the action potential conforms to the “all-or-nothing law”, that is, as long as the stimulus reaches the threshold, it can trigger an action potential.) and a delay in the time to reach the peak value. A delay in the time to reach the peak value of the action potential can reduce the frequency of the action potential, thereby alleviating pain. When the action potential decreases below the threshold potential (when the depolarization of the membrane potential reaches a certain critical value, a large number of Na⁺ channels on the membrane will open, and a large influx of Na⁺ will occur, generating an action potential. This critical value of the membrane potential is called the threshold potential.), the action potential cannot be propagated between nerve cells, thereby blocking the conduction of pain through the nervous system. A decrease in potassium ion concentration will lead to an increase in the resting potential, which in turn will cause an increase in the threshold potential. An increase in the threshold potential makes it more difficult to initiate an action potential. From this, it can be known that the reduction of sodium ions or potassium ions in acupoints or blood can play an analgesic role.

### SUMMARY OF THE INVENTION

### Technical Problems

The current acupuncture anesthesia methods have the shortcomings of unsatisfactory analgesic effect and insufficient duration of pain relief. This is because the human body capacitance is not large enough, so the ability of acupuncture anesthetists and patients to transport electrons through the human body and acupuncture needles to the patient's acupoints and blood to neutralize the sodium ions and potassium ions contained in the patient's acupoints and blood is very limited.

### Technical Solutions

The technical problem to be solved by the invention is to overcome the deficiencies of the existing technologies mentioned above, and to provide a controllable electrostatic acupuncture anesthesia device that can improve the anesthesia effect and prolong the anesthesia time.

### Beneficial Effects

The technical problem of the present invention is solved by the following technical solution. This electrostatic acupuncture anesthesia device includes: at least one conductive needle capable of piercing through the skin and entering acupoints or blood vessels of a human or animal body, characterized in that: it also includes an electrostatic generator; The conductive needle is conductively connected with the electrostatic generator, so that the electrons generated by the electrostatic generator can be released within the acupuncture acupoints or blood vessels of the human or animal body through the conductive needle.

The technical problems of the electrostatic acupuncture anesthesia device of the present invention are further solved by the following technical solution.

The conductive needle is a medical or veterinary acupuncture needle or injection needle.

The connection between the conductive needle and the electrostatic generator is a separable and movable connection.

The connection between the conductive needle and the electrostatic generator is a separable and movable connection through a wire.

The conductive needle is made of metal, alloy or high-temperature superconducting ceramic material.

The electrostatic generator is an electrostatic generator with adjustable output power.

The electrostatic generator is provided with an adjustment knob for adjusting the output power.

The beneficial effects of the electrostatic acupuncture anesthesia device of the present invention compared with the existing technology are as follows:

This electrostatic acupuncture anesthesia device scientifically applies the electrostatic generator, which is usually used in the fields of electrostatic dust removal and electrostatic spraying, to the field of medical acupuncture anesthesia, creating a brand-new application for the electrostatic generator. It utilizes the electrons generated by the electrostatic generator to promote the reduction of the concentration of sodium ions and potassium ions outside the nerve cell membrane and in the blood at the acupoints of patients undergoing acupuncture anesthesia. A decrease in sodium ion concentration can lead to a reduction in the amplitude of the action potential and a delay in the time to reach the peak value, thereby alleviating pain. When the action potential decreases below the threshold potential, it cannot propagate between nerve cells, thereby interrupting the conduction of pain through the nervous system.

A decrease in potassium ion concentration can lead to an increase in the threshold potential. An increase in the threshold potential can make it more difficult to initiate an action potential, that is, the occurrence of pain. Therefore, a decrease in the concentration of sodium ions and potassium ions in acupoints and blood can both play a role in analgesia.

Since the capability of an electrostatic generator to produce electrons far exceeds that of human body capacitance, this electrostatic acupuncture anesthesia device may offer better analgesic effects compared to the manual acupuncture anesthesia performed by current anesthetists. As the output power of the electrostatic acupuncture anesthesia device is adjustable, it has higher reliability compared to the manual acupuncture anesthesia performed by current anesthetists. The electrostatic acupuncture anesthesia device of this invention can be widely used in hospital surgeries and has huge market potential.

### BRIEF DESCRIPTION OF THE DRAWINGS

FIG. 1 is a schematic diagram of the structure of the electrostatic acupuncture anesthesia device;

FIG. 2 is a circuit diagram of the electrostatic generator shown in FIG. 1.

### DETAILED DESCRIPTION OF THE BEST EMBODIMENT OF THE INVENTION

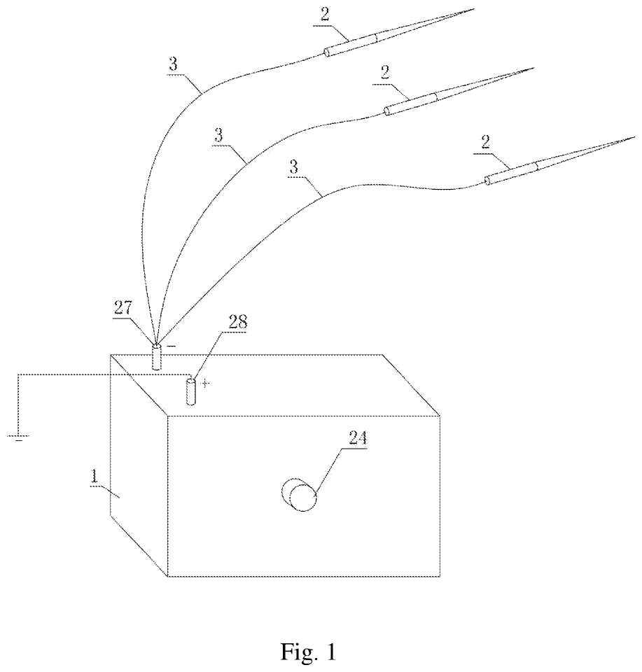

Embodiment: As shown in FIG. 1, the electrostatic acupuncture anesthesia device consists of three conductive needles 1 and an electrostatic generator 2.

The conductive needles 1 are medical acupuncture needles made of metal materials.

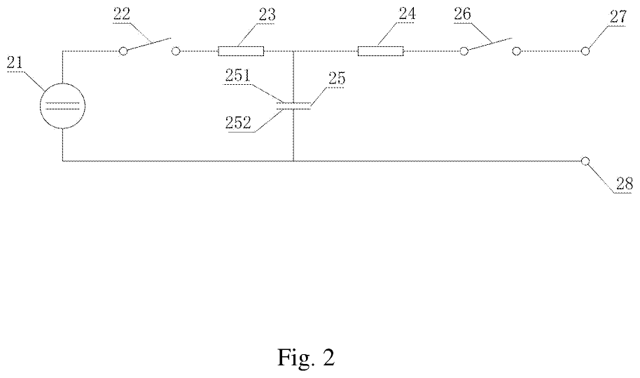

The electrostatic generator 2 is a device capable of generating static electricity and releasing electrons. It usually operates with a single polarity, either positive or negative, and the output voltage can be adjusted. It is commonly used in fields such as electrostatic precipitation and electrostatic painting. The circuit and principle structure of the electrostatic generator 2 are shown in FIG. 2, which consists of a DC power supply 21, a switch 22, a resistor 23, an adjustable resistor 24 with a control knob, a capacitor 25, a switch 26, a cathode 27, and an anode 28.

The non-needle tips of the conductive needles 1 are connected to the cathode 27 of the electrostatic generator 2 through wires 3, so that the electrons generated by the electrostatic generator 2 can be released outward through the needle tips of the conductive needles 1.

When the conductive needles 1 of this device are inserted into the corresponding acupoints of a patient, the electrons generated by the electrostatic generator 2 are released into the patient's acupoints through the conductive acupuncture needles 1 to neutralize the positively charged ions such as sodium ions and potassium ions accumulated in the acupoints, and reduce the concentrations of sodium ions and potassium ions in the patient's blood through the blood circulation, thereby producing an anesthetic effect. The output voltage of the electrostatic generator is adjusted by the variable resistor 24 with a control knob, thereby adjusting the speed of electron release, that is, the output power.

Since the ability of the electrostatic generator 2 to produce electrons is far greater than that of human body capacitance, this electrostatic acupuncture anesthesia device has a better analgesic effect compared with the manual acupuncture anesthesia performed by current anesthetists. And since the output power of this electrostatic acupuncture anesthesia device is adjustable, it has higher reliability compared with the manual acupuncture anesthesia performed by current anesthetists.

During use, first insert the conductive needle into the patient's acupoint or blood vessel and turn on the switch 22 as shown in FIG. 2, allowing the DC power supply 21 to charge the capacitor 25, so that the plate 251 of the capacitor is negatively charged and the plate 252 is positively charged. Then cut off the switch 22, ground the anode 28, and connect the cathode 27 to the conductive needle 1 through the wire 3. Then turn on the switch 26, and the electrons on the plate 251 can be released in the patient's acupoint through the cathode 27 and the conductive acupuncture needle. The output voltage of the electrostatic generator can be adjusted by the variable resistor 24 with a control knob, and gradually reducing the variable resistor 24 can gradually increase the discharge speed of the conductive needle 1 in the patient's acupoint. After a certain period of time, the sodium and potassium ions in the acupoint and blood will decrease to a certain level, at which point the patient begins to enter an anesthetized state. The doctor can then start the surgery on the patient. Since the electrons in the patient's blood will continuously conduct to the patient's body and then conduct outward through the patient's body, the electrostatic generator needs to maintain its operating state during the surgery. After the surgery is completed, turn off the electrostatic generator and remove the conductive needle. The electrons that enter the blood will slowly conduct to the patient's body and then conduct to the outside through the skin, so the patient can automatically recover from the anesthetized state. After the operation, the conductive acupuncture needle can also be switched to the positive electrode of the electrostatic generator (so that the negative electrode is grounded), and the conductive acupuncture needle can be used to neutralize the anion in the patient's blood, so that the patient can recover from the anesthetic state. Then turn off the electrostatic generator and pull out the conductive needle.

The above electrostatic acupuncture anesthesia device can also be used in animals, and the diameter and length of the conductive needle used in the electrostatic acupuncture anesthesia device for large animals are obviously larger than the electrostatic acupuncture anesthesia device for people. For example, acupuncture anesthesia devices used in elephants can have a conductive needle diameter of 2-5 mm.

The above content is a further detailed explanation of the present invention in combination with a specific preferred embodiment, and it cannot be considered that the specific implementation of the present invention is limited only to these descriptions. For those skilled in the technical field of the present invention, under the premise of not departing from the conception of the present novel practical type, its structure form can be flexibly changed, and a series of products can be derived. Merely making some simple deductions or replacements should all be regarded as belonging to the patent protection scope determined by the claims submitted for the present invention.

What is claimed is:

1. An electrostatic acupuncture anesthesia device, comprising: at least one conductive needle which can be pierced into the acupoint or blood vessel of human body or animal body, wherein further comprises an electrostatic generator; the conductive needle is in conductive connection with the electrostatic generator, so that the electrons generated by the electrostatic generator can be released in the punctured acupoint or blood vessel of the human body or animal body through the conductive needle.

2. The electrostatic acupuncture anesthesia device according to claim 1, wherein the conductive needle is a medical or veterinary acupuncture needle or injection needle.

3. The electrostatic acupuncture anesthesia device according to claim 2, wherein the conductive needle and the electrostatic generator are separably and movably connected.

4. The electrostatic acupuncture anesthesia device according to claim 3, wherein the conductive needle and the electrostatic generator are separably and movably connected through a wire.

5. The electrostatic acupuncture anesthesia device according to claim 4, wherein the conductive needle is made of metal, alloy or high-temperature superconducting ceramic material.

6. The electrostatic acupuncture anesthesia device according to claim 5, wherein the electrostatic generator is an electrostatic generator with adjustable output power.

7. The electrostatic acupuncture anesthesia device according to claim 6, wherein the electrostatic generator is provided with a variable resistor with a control knob which can adjust the output voltage of the electrostatic generator.
